(19) United States
(12) Patent Application Publication
(10) Pub. No.: US 2024/0226457 A1
(43) Pub. Date: Jul. 11, 2024

(54) Medical Syringe

(71) Applicants: Thomas J. Shaw, Frisco, TX (US); Retractable Technologies, Inc., Little Elm, TX (US)

(72) Inventors: Thomas J. Shaw, Frisco, TX (US); Mark Small, Heavener, OK (US)

(21) Appl. No.: 18/616,683

(22) Filed: Mar. 26, 2024

Related U.S. Application Data
(63) Continuation-in-part of application No. 17/518,308, filed on Nov. 3, 2021.
Application No. 17/518,308 is a continuation-in-part of application No. 16/370,239, filed on Mar. 29, 2019.
Application No. 16/370,239 is a continuation-in-part of application No. 15/940,305, filed on Mar. 29, 2018.

(51) Int. Cl. A61M 5/32

(52) U.S. Cl. CPC A61M 5/3216

(57) ABSTRACT

A safety syringe for medical use is disclosed as having an injection-molded body with a tubular barrel having a finger flange projecting transversely from the barrel near a proximal end of the barrel and a cylindrical collar projecting rearwardly from the finger flange to the proximal end of the barrel. A plunger assembly includes a plunger handle end cap and an elastomeric plunger seal insertable into the barrel through a rearwardly facing opening in the cylindrical collar to slidably engage the cylindrical inside wall of the barrel. A cylindrical elastomeric bushing is frictionally engaged around an outside wall of the cylindrical collar, and a selectively removable plunger cap frictionally engages the cylindrical elastomeric bushing. A safety syringe may also include a needle safety device.

### CROSS-REFERENCE TO RELATED APPLICATIONS

This application is a continuation-in-part of U.S. application Ser. No. 17/518,308, filed Nov. 3, 2021, which is a continuation-in-part of U.S. application Ser. No. 16/370,239, filed Mar. 29, 2019, which is a continuation-in-part of application Ser. No. 15/940,305, filed Mar. 29, 2018.

### BACKGROUND OF THE INVENTION

### 1. Field of the Invention

This invention relates to a safety syringe configured for medical use in aspirating or injecting fluids that does not comprise a retractable needle. As used here, the term “safety syringe” refers to a syringe having one or more structures and/or protection features that function or cooperate to reduce the likelihood that a user or subsequent handler of the device, a patient or a bystander will experience an inadvertent, accidental or unintended needle stick from a needle or cannula that is part of or used in association with the subject device.

### 2. Description of Related Art

Syringes intended for medical use are typically made with removable plunger caps that help prevent contaminants from entering a syringe barrel through the annular space that otherwise exists around the plunger handle at the rear of the barrel prior to use of the device. Such plunger caps are typically removed and disposed of just prior to use of the syringe.

Syringes intended for medical use are typically made with barrels comprising substantially cylindrical inside and outside walls, meaning that volumetric dosage indicia or other markings are applied to an arcuate outer surface during manufacture. This can be difficult, and especially when the diameter of the barrel and radius of curvature of the outside wall are small (as with 1 mL, 0.5 mL and smaller syringes) and where the available outside surface area is extremely limited, or on syringes where different dosing scales or other indicia are applied to opposite sides of the barrel. In such instances it is often necessary to spin or rotate the barrel while printing, and it is also often difficult to read indicia such as dose measuring lines and the related numeric values or other markings because they wrap so far around the circumference of the barrel. This problem is accentuated with small-diameter, low-dose syringes where the dosage number is less than “1” and must be preceded by a “0.” In such cases, the principal digit identifying the dosage associated with an injection is often not visible when focusing on the volumetric measuring indicia to determine the fluid level within the syringe. These design deficiencies can confuse the user, particularly older users with poor or compromised vision, and contribute to the commission of medical errors.

Other design deficiencies exist with some so-called “safety syringes” that have needle shields or guards that are rotated into place following an injection. Prior to and during an injection, such shields or guards often impede or block a significant portion of the dosage scale and indicia, thereby also contributing to the commission of medical errors.

Other design deficiencies exist with some “safety syringes” having longitudinally extending hubs or other slidable safety elements that increase the overall length of a syringe and contribute to the “dead space” within a syringe. Syringes are needed that protect users, patients and bystanders from accidental needle sticks without also increasing the amount of dead space. A reduction in the amount of dead space within a syringe is necessary to reduce medicinal waste and increase the number of doses available from a single vial. Other design deficiencies exist with respect to syringes having flat indicia-bearing surfaces configured as sleeves or clips attachable to a syringe barrel.

For at least these reasons, low-dose safety syringes with barrels having integrally molded, easily viewable, printable surfaces with factory-applied dosage markings and other indicia are needed. Such syringes will desirably embody low dead space, protect users and patients against accidental needle-stick injuries, and be manufacturable using high speed injection molding methods and equipment that also reduce the per unit cost of such devices.

During the recent and ongoing worldwide pandemic associated with corona virus and other virulent pathogens, increased demand exists for safety syringes that can be made quickly and reliably in various sizes and needle gauges. At the same time, there is increased emphasis on the need for improved syringe designs that can be manufactured more efficiently and inexpensively, with lower material and packaging costs. A syringe is needed that combines a safe and reliable needle-stick protection feature with integrally molded, flat and printable indicia display surfaces and a plunger assembly. Also needed is a safety syringe body embodying the foregoing figures that does not require use of a slide for molding undercuts and is instead configured to provide clearance for core pins during injection molding of the syringe body to facilitate use of sixty-four cavity molds with an attendant increase in production capacity and reduction in production costs.

### SUMMARY OF THE INVENTION

A satisfactory embodiment of the subject safety syringe desirably comprises an injection-molded plastic body further comprising a tubular barrel with a needle holder projecting forwardly at its distal end, a transversely projecting finger flange disposed near its proximal end and a cylindrical collar projecting rearwardly from the barrel behind the transversely projecting finger flange, and two longitudinally extending wings contiguous with and projecting upwardly from the barrel in parallel and spaced-apart relation. Each wing has an inwardly facing, integrally molded support and guide rail slidably engaging a cooperatively sized and aligned, longitudinally extending channel of a needle safety shield. Each wing desirably further comprises a substantially flat, outwardly facing surface bearing an array of volumetric marking indicia and dosage numbers disposed proximally to a lower portion of the barrel to be easily readable by a user. The wings each have a proximal end that is contiguous to a side of a window disposed above the barrel in the transverse finger flange. The window, not present in the finger flanges of known prior art devices, is provided to allow insertion and retraction of a core pin between the wings during molding to facilitate more efficient molding and thereby reduce associated production time and product costs.

In some instances in this disclosure, the two wings of the body may be said to project “tangentially” from the barrel. As used in such instances, “tangentially” means that the two elongate wings extend generally upward from opposite sides of the upwardly facing portion of the barrel rather than extending laterally outward from the barrel as with wings used to stabilize some prior art medical devices such as catheter introducers against a body part of a patient. It will be appreciated by those skilled in the art of injection molding such plastic devices as the body of the subject safety syringe that modifications to design criteria such as draft angles and radii of curvature may be needed that do not strictly comport with a mathematical definition of “tangential.” Such modifications may be needed, for example, to facilitate molding and reduce stress concentrations along the bases of each wing where they are integrally molded as part of the barrel portion of the body. Similar design modifications may be needed where each wing is integrally molded to its respective guide rail.

A forwardly projecting needle is desirably attached in fixed relation to the needle holder disposed at the distal end of the barrel and a selectively releasable needle cover is desirably provided to protect the needle tip and guard against inadvertent needle-stick injuries or contamination prior to use of the device. The needle cover desirably has a closed distal end and a proximal open end that frictionally engages a forwardly extending nose of the barrel.

The subject invention also desirably comprises a plunger assembly further comprising an elongate handle that is insertable into the barrel through an opening provided by the cylindrical collar projecting rearwardly behind the transverse finger flange of the body. An elastomeric plunger seal disposed at the distal end of the plunger handle slidably engages an inside wall of the tubular barrel. The proximal end of the plunger handle desirably comprises a circular solid end cap having an outer diameter slightly greater than that of the barrel. When the plunger handle is advanced inside the barrel, the forwardly facing annular surface of the plunger handle end cap desirably abuts against a portion of the rearwardly facing annular surface of the barrel collar that extends rearwardly of the finger flange and is configured to receive a slidably attached, selectively releasable plunger cap.

To better accommodate the smaller syringe diameters achievable with use of flat indicia display surfaces instead of printing on the barrel wall as with conventional syringes, a new cylindrical retainer bushing is desirably provided around the rearwardly projecting barrel collar forwardly of the plunger handle end cap. The cylindrical retainer bushing (or sleeve or washer) is desirably configured to provide a frictionally engageable seating and sealing surface for the releasably attachable plunger cap as the plunger cap and is preferably made of an elastomeric material.

When the needle cover is installed and the plunger is advanced inside the barrel so that the forwardly projecting cylindrical collar of the plunger abuts against the rearwardly facing surface of the transverse finger flange, the internal, fluid-contacting portions of the syringe are enclosed and protected from contamination whether or not the syringe is also enclosed inside another package. Because of this, the syringes can be assembled and shipped in bulk prior to packaging and sterilization.

The longitudinally extending needle safety shield is disposed above and in parallel alignment with the tubular barrel. The needle safety shield desirably comprises an activation handle with oppositely facing elongate channels that are cooperatively configured and aligned to provide longitudinal sliding engagement with the support and guide rails integrally molded on the inwardly facing surfaces of the wings. The activation handle also desirably comprises an upwardly facing textured touch surface that facilitates the application of manual pressure to initiate forwardly sliding movement of the needle safety shield to reposition a cylindrical needle guard disposed at its distal end forwardly into a position that surrounds the needle tip following use to protect patients and medical personnel from accidental needle sticks and the attendant risk of infection by blood-borne pathogens. The cylindrical needle guard is desirably seated around a forwardly facing cylindrical nose of the barrel during storage and use of the device prior to forward movement of the activation handle. The longitudinal support and guide rails of the wings also desirably cooperate with interference elements, ramps, and shoulder stops molded on the inwardly facing surfaces of the wings to restrict sliding movement of the sliding safety shield relative to the barrel when needed during use of the device, as further described below. A principal advantage of the subject invention is a body configuration having a window through the finger flange that enables the insertion and retraction of core pins to mold the undercut portions of interference elements, ramps and stops of the body without the need for a slide.

Each wing of the body has a base that is contiguous with and projects upwardly from the barrel and comprises an outwardly facing, substantially flat indicia display surface bearing printed dosage markings. The two oppositely disposed wings can have the same or different dosage scales to facilitate volumetric measurements marked in different units. The subject invention is particularly useful with syringes having usable volumes of 1 mL or less, which syringes typically have smaller barrel diameters that cause the volumetric dosage indicia applied to the outside surface to wrap around a greater portion of the circumference of the barrel. According to one embodiment of the invention, pad printing technology (sometimes referred to as “tampography”) is used to apply volumetric dosage indicia or other markings to the substantially flat display surface of the syringe. As used here, the term “substantially flat” refers to the general configuration of the relevant surface and can include a slight curvature, draft or minor undulations with comparatively minor raised or recessed portions or features, and particularly, those features attributable to the applied indicia or other markings.

The subject safety syringe embodies needle protection features necessary for safety considerations without the need for and incremental expense of a retractable needle. This embodiment of the invention combines the advantages of flat, printable surfaces having more easily readable dosage indicia with the cost advantages of a simpler but still effective and easy-to-use needle safety device to provide a safe and more affordable solution, particularly for widespread administration of injections associated with vaccines, flu shots, and treatments for persons needing frequent injections (e.g., insulin) to treat various chronic health conditions or for those living in underdeveloped locations. Furthermore, because the dosage markings and indicia are more easily readable by the user, there is less risk of administering an incorrect dosage (“medical error”) of a medicinal fluid to a patient and thereby less risk of causing other unintended consequences.

The subject safety syringe desirably embodies wider, substantially flat display surfaces disposed on a medical syringe that cooperate with substantially flat edge portions of the surrounding transverse finger flange to help prevent the syringe from rolling off a tray or other flat surface. The substantially flat surfaces also allow the flange around the barrel to be proportionally narrower as compared to the flanges of conventional syringes having tubular barrels and still provide larger surface areas that are more easily graspable by a user. The oppositely facing, substantially flat wing surfaces also improve stability and the degree of control that can be exercised over the syringe by a user during an injection or other procedure.

The subject safety syringe is also desirably characterized by a low amount of “dead space” between the plunger and needle as compared, for example, to other prior art and competitive syringe configurations. As used here, the term “dead space” refers to the interior volume occupied by medicinal fluid that remains inside a syringe when the plunger is fully advanced inside the barrel. Excessive dead space contributes to medicinal waste during an injection and can also contribute to the formation of undesirable air bubbles inside the fluid chamber of a syringe during aspiration of a fluid. Use of syringes having low dead space can in some cases enable users to obtain an additional dose from each vial of medication. Although the subject syringe is especially useful in administering relatively small doses of a medicinal fluid such as insulin or a vaccine to a user by injection or infusion, the structure and operation of the apparatus is not limited to particular sizes, doses or procedures.

Another embodiment of the invention is disclosed that desirably embodies one and preferably two oppositely facing, substantially flat indicia display surfaces and also embodies a needle safety shield having a needle tip guard that extends circumferentially around the needle and is desirably coaxially aligned with the needle. The needle tip guard is attached or connected to, or unitarily molded together with, an activation handle that slidably engages the body. Suitable rails, ramps, stop shoulders and detents or other similarly effective means are desirably provided as part of the needle safety device and the barrel so that the activation handle can be advanced smoothly and without substantial interference when desired, and will not accidentally retract afterward to expose the needle tip. The needle safety device of this embodiment of the invention eliminates the need for having a needle retraction mechanism, a needle retraction cavity or any transverse sliding movement of the barrel relative to the needle to protect users from accidental needle sticks. Following use of the syringe, the needle tip guard is selectively advanced to protect the user from the forwardly projecting needle tip by applying manual pressure to a touch surface of the activation handle that is located rearwardly of the needle and needle tip guard. The activation handle slidably engages the syringe body and is forwardly slidable relative to the barrel from a first position in which the needle tip is uncovered to a second position in which the needle tip is surrounded and protected against inadvertent needle sticks by the needle tip guard. The needle tip is desirably disposed sufficiently inside the needle tip guard once the needle safety device is fully extended relative to the barrel that someone handling the used syringe will not inadvertently be subjected to a needle stick injury by simply placing a fingertip over the end of the syringe.

In another embodiment of the invention, the barrel and the activation handle comprise cooperatively engageable elements that enable manually actuated and controllable, longitudinally slidable movement of the activation handle relative to the barrel between two predetermined stop positions. The first stop position is a fully retracted use position in which the needle tip guard substantially surrounds the nose. The second stop position is a fully extended post-use position in which the needle tip shield circumferentially surrounds a tip end of the forwardly projecting needle.

In one preferred embodiment of the invention, the forwardly projecting needle is disposed in fixed relation to the nose of the barrel but it will be appreciated upon reading this disclosure that a similarly configured syringe can be made with needles configured to be selectively attachable to the barrel such as by the use of threads or other similarly effective means.

These and other features of the present invention will be better understood from a consideration of the following detailed description of various embodiments and appended claims in conjunction with the accompanying drawings.

### BRIEF DESCRIPTION OF THE DRAWINGS

The syringe of the invention is further described and explained in relation to the following drawings wherein:

FIG. 1 is a top front perspective view of a safety syringe for medical use according to an embodiment of the disclosure in which the removable needle cap and plunger end cap are installed in the position in which a plurality of the subject syringes can be packaged, shipped and stored, and the plunger is fully advanced inside the barrel;

FIG. 2 is a top rear perspective view of the safety syringe according to an embodiment of the disclosure;

FIG. 3 is an exploded top front perspective view of the safety syringe according to an embodiment of the disclosure;

FIG. 4 is a top front perspective view of the safety syringe according to an embodiment of the disclosure with the needle cap and plunger end cap removed, the plunger fully advanced relative to the body, and the needle safety shield disposed in its pre-use (retracted) position;

FIG. 5 is a orthogonal side view of the safety syringe according to an embodiment of the disclosure with the needle cap and the plunger end cap in place;

FIG. 6 is a cross-sectional side view of the safety syringe according to an embodiment of the disclosure;

FIG. 7 is a top rear perspective view of the safety syringe according to an embodiment of the disclosure with the needle cap and plunger end cap removed, and the plunger partially withdrawn relative to the body and the needle safety shield disposed in its pre-use position;

FIG. 8 is a cross-sectional side view of the safety syringe according to an embodiment of the disclosure with the needle cap and plunger end cap removed, the plunger partially withdrawn relative to the body, and the needle safety shield disposed in its pre-use (retracted) position;

FIG. 9 is a top front perspective view of the safety syringe according to an embodiment of the disclosure with the needle cap and plunger end cap removed and the plunger fully advanced inside the barrel with the needle safety shield advanced to its post-use “safety” position; and

FIG. 10 is a cross-sectional side view of the safety syringe according to an embodiment of the disclosure with the needle cap and plunger end cap removed, the plunger fully advanced relative to the body, and the needle safety shield disposed in its post-use (extended) position.

### DESCRIPTION OF PREFERRED EMBODIMENTS

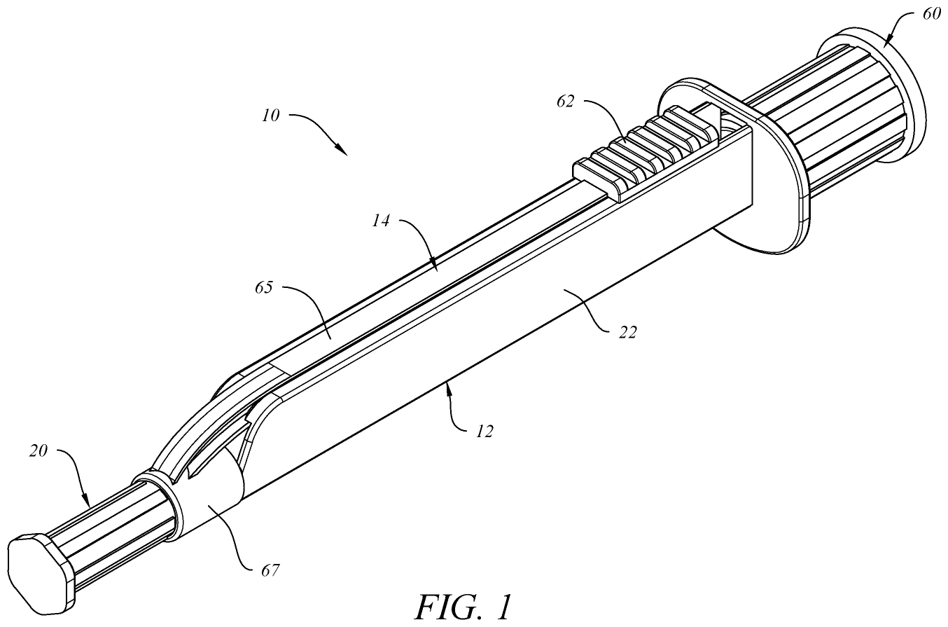

Referring to FIGS. 1-3, safety syringe 10 comprises body 12, needle safety shield 14, plunger assembly 16, needle 18 and removable needle cap 20. Body 12 is desirably molded from a medical grade polymeric material and is sufficiently transparent to permit the liquid level drawn into barrel 22 to be plainly viewed by a user. Body 12 further comprises two upright, substantially parallel, spaced apart wings 24, 26 projecting tangentially above barrel 22 that define an elongate, axially extending space 28 between them. The outwardly facing surfaces of at least one, and preferably both, wings 24, 26 are desirably substantially flat and can comprise an array of volumetric measuring indicia (not shown) applied to each display surface. Such arrays can include, for example, at least the Arabic numerals identifying the number of fluid units and the associated principal measurement indicia. At least a portion of the indicia or markings desirably commences on the substantially flat, outwardly facing display surface of walls 24, 26 and optionally continues for a short distance onto the curved outer wall of barrel 22. Such placement is understood to be within the operational capabilities of conventional pad printing technology that is now readily available to those of ordinary skill in the art.

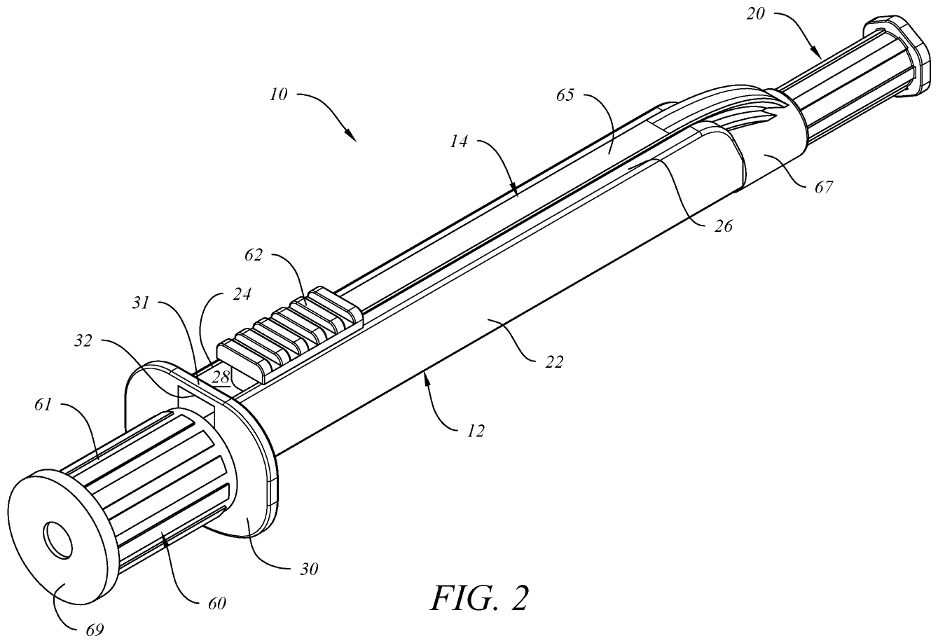

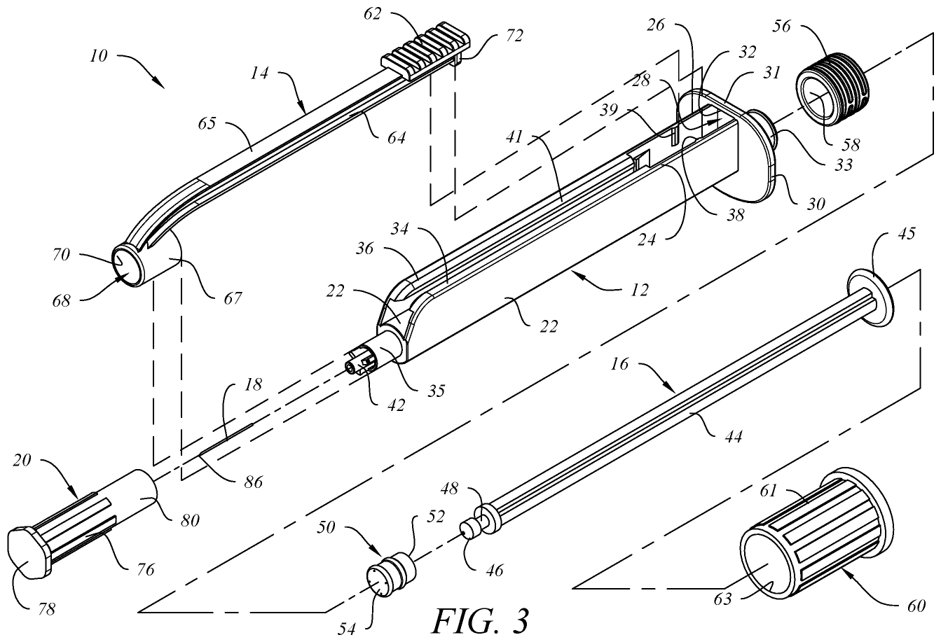

Finger flange 30 is disposed transversely to barrel 22 and projects outwardly around barrel 22 and the proximal end of wings 24, 26. Finger flange 30 also comprises window 32 having a bottom contiguous with the top surface of barrel 22, sides contiguous with the proximal ends of the inwardly facing surfaces of wings 24, 26 and a top side 31 (FIGS. 1-2) that bridges over the top of window 32 and provides structural support and reinforcement to the upwardly projecting proximal ends of wings 24, 26. Cylindrical collar 33 (FIGS. 3, 6) extends rearwardly from finger flange 30 and provides a rearwardly facing opening that is coaxially aligned and communicates with the inside wall of barrel 22. Body 12 of safety syringe 10 further comprises cylindrical nose 35 projecting forwardly from barrel 22 and needle holder 42 projecting forwardly from cylindrical nose 35.

Needle safety shield 14 desirably comprises elongate activation handle 65 configured to fit between and slidably engage upright walls 24, 26 of body 12. Axially extending channels 64 (FIG. 3) are desirably provided on each side of activation handle 65 to slidably engage cooperative sized and aligned support and guide rails 34, 36 that project inwardly from the inwardly facing surfaces of wings 24, 26, respectively. Needle safety guide further comprises an upwardly facing, desirably textured, touch pad 62 to facilitate the application of digital pressure needed to selectively reposition the slidably engaged needle safety shield from a first position to a second position following use of the device. A forwardly extending portion of activation handle 65 desirably curves downwardly and is attached to an integrally molded, cylindrical needle guard 67 comprising an inside wall 70 defining an interior passage 68. Inside wall 70 of cylindrical needle guard 67 is desirably sized and aligned to slidably engage the outwardly facing surface of cylindrical nose 35 of body 12 when channels 64 of activation handle 65 of needle safety shield 14 receive and slidably engage inwardly facing support and guide rails 34, 36 of body 12 during assembly of safety syringe 10.

Referring to FIG. 3, plunger assembly 16 comprises elongate plunger handle 44 having integrally molded thumb cap 45 at its proximal end and distal end projection 46 and recessed annular seating surface 48 for elastomeric plunger seal 50. Elastomeric plunger seal 50 further comprises rear collar 52 attachable to distal end projection 46 and forwardly facing end surface 54. Referring to FIGS. 3, 5 and 6, the configuration of elastomeric plunger seal 50 is desirably such that it provides a fluid seal against the inside wall of barrel 22 when the distal end of plunger handle 44 is inserted forwardly through rearwardly projecting cylindrical collar 33 of barrel 22 of body 12 and into sliding engagement with the inside wall of barrel 22.

In some embodiments, the plunger assembly 16 may comprise a color that is different from a color from other portions of the syringe 10. For example, in some embodiments, the plunger assembly 16 may comprise a blue color, and the cylindrical collar 33 may comprise a white color. A camera or other imaging device (which may be disposed in a mobile device) may be used to capture an image of the plunger assembly 16 (or the thumb cap 45) with respect to the cylindrical collar 45 (or the barrel 22). The image may be relayed to a software application installed on the mobile device or in communication wirelessly with the mobile device, where the software application, where the differing colors may allow the software application to differentiate between the components and determine a relative position of the plunger assembly 16 within the barrel 22 and/or to the cylindrical collar 33. In some embodiments, the relative positions captured by the imaging device may be analyzed by the software application to determine the volume of a medicinal fluid in the barrel 22 and/or the syringe 10. Further, it will be appreciated that any two different colors may be used. For example, in some embodiments, one component (such as the plunger assembly 16 may be orange, while the other component (such as the barrel 22 or the cylindrical collar 33) may be white or transparent.

Still referring to FIGS. 3, 5 and 6, molded plastic plunger end cap 60 having cylindrical skirt 61 with open distal end 63 and a closed or partially closed proximal end 69 is desirably provided to protect against contamination and reduce the likelihood of accidental premature withdrawal of plunger assembly 16 from barrel 22 during shipment and storage of safety syringe 10 prior to use. An elastomeric bushing (or sleeve or washer) 56 preferably having one or more textured cylindrical surfaces is desirably disposed between the outwardly facing wall of rearwardly projecting cylindrical collar 33 and the inwardly facing wall of skirt 61 of plunger end cap to releasably hold plunger end cap 60 in place relative to body 12 of safety syringe 10 prior to use. The respective diameters of cylindrical collar 33, elastomeric bushing 56 and skirt 61 of plunger end cap 60 and the thickness, surface texture and durometer of elastomeric bushing 56 are desirably selected and cooperatively configured to provide a releasable frictional holding force sufficient to maintain plunger end cap 60 in stable relation to each other and to safety syringe 10 prior to use.

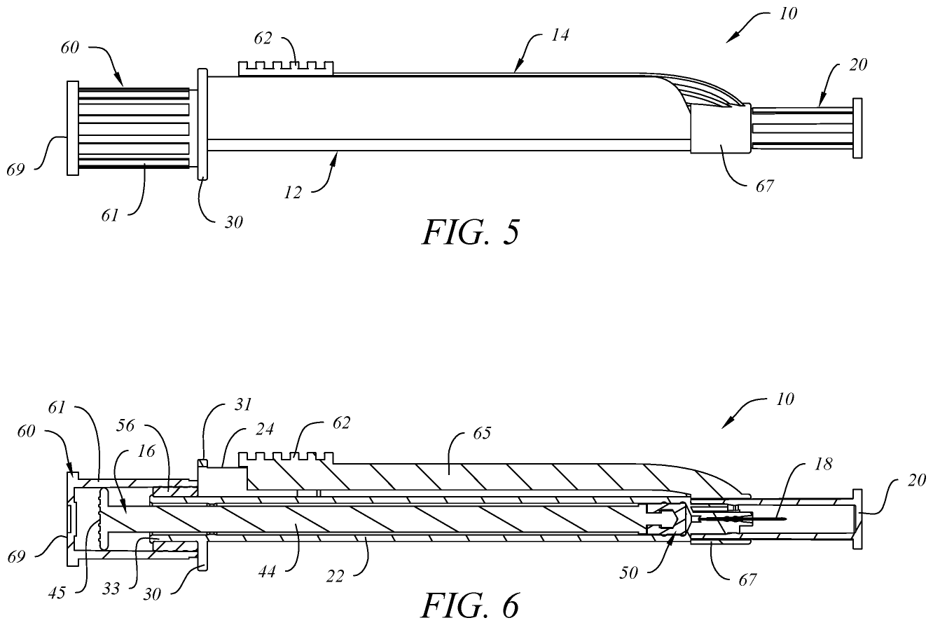

In some embodiments, elastomeric bushing 56 may be configured to slide forwardly over rearwardly projecting cylindrical collar 33 of barrel 22 when plunger seal 50 and plunger handle 44 are inserted forwardly into barrel 22 during assembly of safety syringe 10 to the configuration depicted in FIGS. 5 and 6. The rear end of needle 18 is insertable into needle holder 42 and attached in fixed relation to the needle holder such as by using an adhesive or another similarly effective, commercially available means.

Referring to FIGS. 5 and 6, safety syringe 10 is fully assembled with needle cap 20 covering forwardly projecting needle 18 having front needle tip 86, and plunger handle disposed fully forward inside barrel 22 so that cylindrical collar 61 projecting forwardly from plunger end cap 60 is abutting against the rearwardly facing surface of finger flange 30.

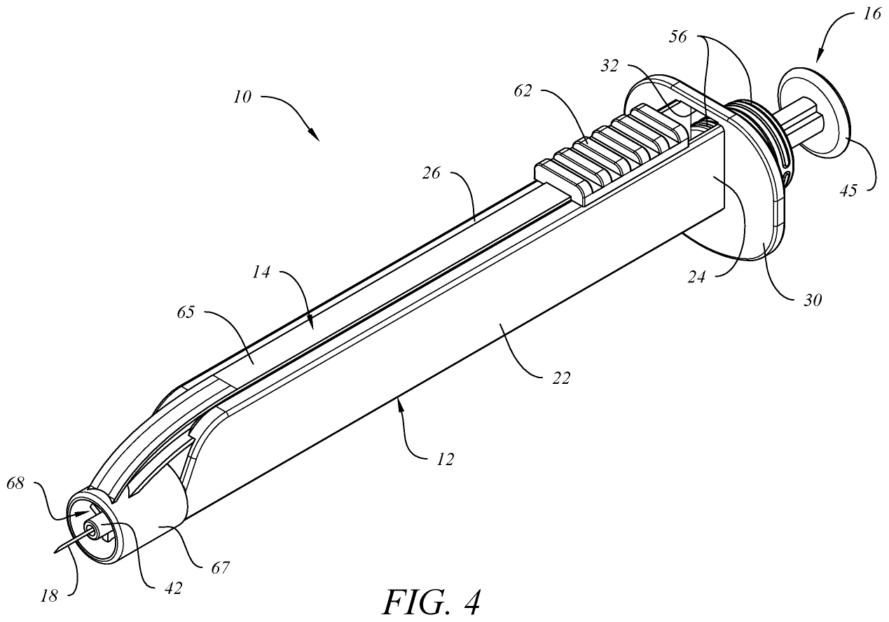

Referring to FIG. 4, safety syringe 10 is depicted in the “pre-use” position with needle cap 20 (FIGS. 1-2) removed, needle 18 projecting forwardly from needle holder 42 of barrel 22, and with needle safety shield 14 in its first stop position with cylindrical needle guard 67 disposed in its pre-use position around needle holder 42 and cylindrical nose 35 (FIG. 3) of body 12. Referring to FIGS. 1, 3, 6 and 11, the diametric clearance between the outwardly facing surface of cylindrical nose 35 and the inside wall 70 of cylindrical needle guard 67 is desirably sufficient to allow the rearwardly extending cylindrical portion 80 of selectively removable needle cap 20 to be inserted between them and to frictionally engage the outside surface of cylindrical nose 35. The length of sidewall section 76 of needle cap 20 is desirably sufficient to accommodate needles (such as needle 18) of various lengths and gauges without allowing needle tip 86 (FIGS. 6) to engage solid end cap 78 prior to use of safety syringe 10. As seen in FIG. 11, little dead space 84 is present between plunger seal 50 and the proximal end of needle 18 inside needle holder 42 when plunger handle 44 is fully advanced inside barrel 22.

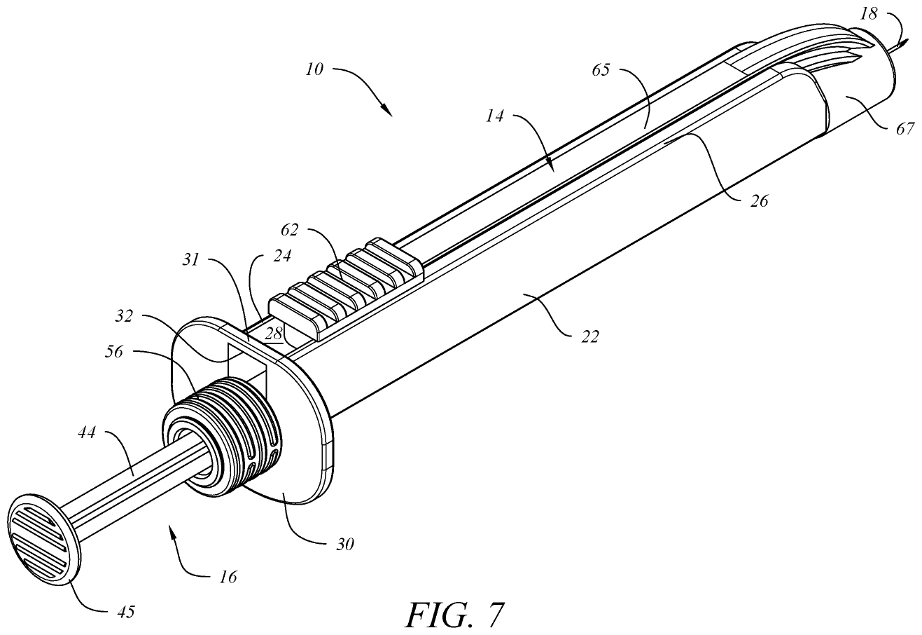

Referring next to FIGS. 4 and 7, after needle cap 20 is removed, and with needle safety shield 14 still disposed in its first stop position with cylindrical needle guard 67 still disposed around needle holder 42 and cylindrical nose 35 of body 12, needle 18 can be inserted into a medicine vial and plunger cap 60 of plunger assembly 16 can be withdrawn a desired distance relative to barrel 22 to create a fluid chamber 82 inside barrel 22 to aspirate an injection.

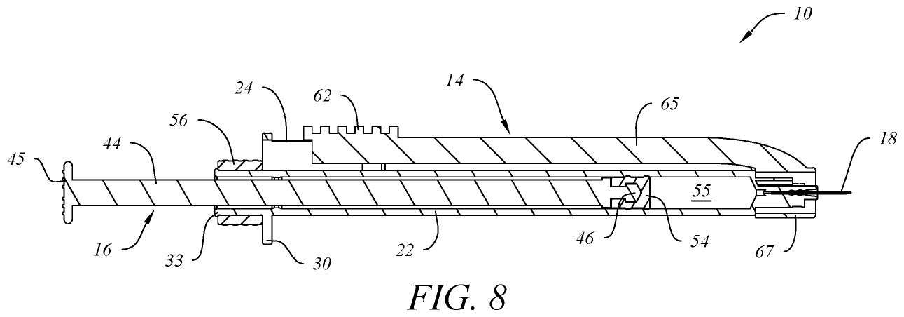

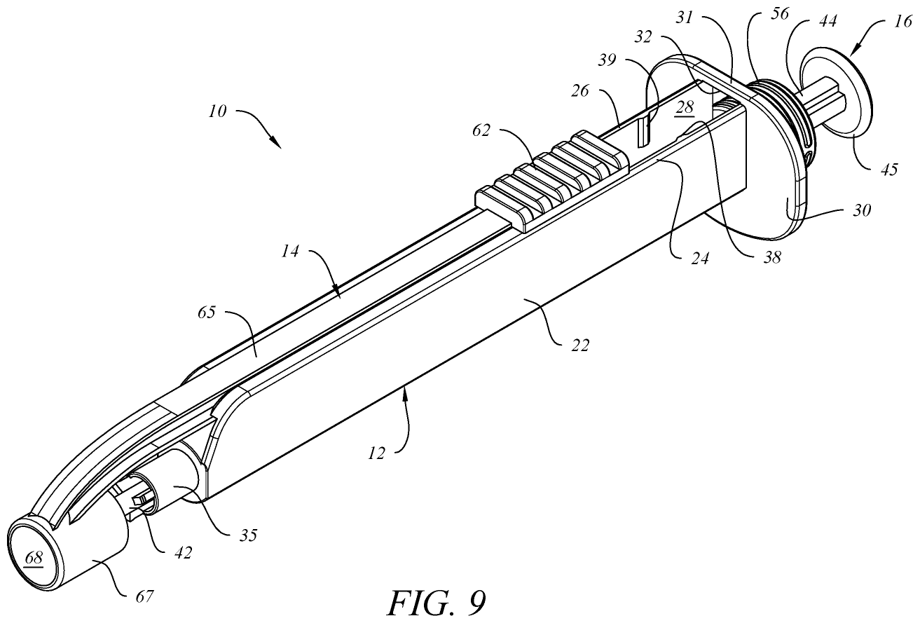

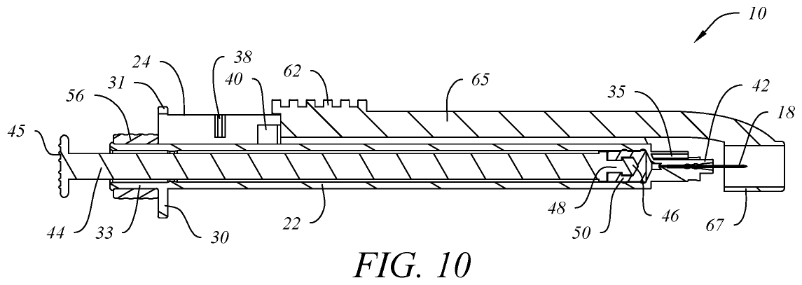

Following the injection, during which plunger handle 44 is again fully advanced inside barrel 22 as shown in FIGS. 8-10, light manual pressure is desirably applied to upwardly facing touch surface 62 of activation handle 65 of needle safety shield 14 to initiate forward movement of needle safety shield 14 in elongate space 28 relative to barrel 22 and wings 24, 26 of body 12.

It will be appreciated that a safety syringe for medical use may comprise one or more of the following embodiments:

Embodiment 1. A safety syringe for medical use, comprising: an injection-molded body comprising a tubular barrel having a cylindrical inside wall, a proximal end, and a distal end, a cylindrical nose comprising a needle holder comprising a needle coupled to and projecting forwardly from the needle holder at the distal end of the barrel, a finger flange projecting transversely from the barrel near the proximal end, a cylindrical collar projecting rearwardly from the finger flange to the proximal end of the barrel, and two upright wings disposed and extending forwardly from the finger flange in a parallel and spaced-apart relationship above the barrel; a plunger assembly comprising a plunger handle having a proximal end and a distal end, the proximal end having a plunger handle end cap and the distal end having an elastomeric plunger seal insertable into the barrel through a rearwardly facing opening in the cylindrical collar to slidably engage the cylindrical inside wall of the barrel; a cylindrical elastomeric bushing frictionally engaging an outside wall of the cylindrical collar; a selectively removable plunger cap frictionally engaging the cylindrical elastomeric bushing; and a needle safety shield disposed between the two upright wings and slidably aligned with the barrel.

Embodiment 2. The safety syringe of embodiment 1, wherein the needle safety shield comprises an activation handle having a proximal end and a distal end, the activation handle slidably engaging the barrel and having an upwardly facing touch surface that facilitates application of manual pressure by a user to initiate forward sliding movement of the needle safety shield relative to the barrel.

Embodiment 3. The safety syringe of embodiment 2, wherein the forward sliding movement of the needle safety shield repositions a cylindrical needle guard disposed at the distal end of the needle safety shield from a first position surrounding the cylindrical nose of the barrel during storage and use of the safety syringe to a second position guarding a needle tip of the needle following use of the safety syringe.

Embodiment 4. The safety syringe of embodiment 3, wherein each of the two upright wings comprises an inwardly projecting, longitudinally extending guide rail to which the activation handle of the needle safety shield slidably engages.

Embodiment 5. The safety syringe of embodiment 4, wherein each longitudinally extending guide rail is cooperatively sized and aligned with a channels disposed on opposing sides of the activation handle of the needle safety shield, and wherein each of the channels slidably engages the respective guide rail.

Embodiment 6. The safety syringe of embodiment 5, wherein each support and guide rail is integrally molded as part of one of the two upright wings from which it projects.

Embodiment 7. The safety syringe of embodiment 1, wherein the finger flange comprises a window.

Embodiment 8. The safety syringe of embodiment 7, wherein the window is cooperatively sized and aligned with an activation handle of the needle safety shield that is disposed between the two upright wings.

Embodiment 9. The safety syringe of embodiment 7, wherein the window comprises a first side that is contiguous with a proximal end portion of an inwardly facing wall of each of the two upright wings.

Embodiment 10. The safety syringe of embodiment 9, wherein the window has a top section that bridges and provides structural support for the two upright wings.

Embodiment 11. The safety syringe of embodiment 1, wherein at least one of the two upright wings comprises a substantially flat, outwardly facing indicia display surface configured to bear indicia related to volumetric measurements of a fluid disposed inside the syringe.

Embodiment 12. The safety syringe of embodiment 11, wherein each of the two upright wings comprises a substantially flat, outwardly facing surface bearing indicia related to volumetric measurements of a fluid disposed inside the syringe.

Embodiment 13. The safety syringe of embodiment 1, wherein the two upright wings comprise one or more interference elements molded on inwardly facing surfaces of the two upright wings to restrict sliding movement of the needle safety shield relative to the barrel.

Embodiment 14. The safety syringe of embodiment 13, wherein each of the one or more interference elements comprise a ramp having a forwardly facing stop shoulder.

Embodiment 15. The safety syringe of embodiment 14, wherein the needle safety shield comprises a rearwardly facing stop surface that engages forwardly facing stop shoulders of the ramps to prevent subsequent rearward movement of the needle safety shield relative to the barrel following repositioning of the needle safety shield from a first position to a second position.

Embodiment 16. The safety syringe of embodiment 1, wherein a cylindrical needle guard disposed at the distal end of the needle safety shield surrounds the cylindrical nose of the barrel during storage and use of the safety syringe, and wherein the cylindrical needle guard envelops a needle tip of the needle following use of the safety syringe.

Embodiment 17. The safety syringe of embodiment 1, wherein the cylindrical collar comprises a first color, and wherein the plunger assembly comprises a second color that is different from the first color.

Embodiment 18. The safety syringe of embodiment 17, wherein the cylindrical collar is white, and wherein the plunger assembly is blue.

Embodiment 19. The safety syringe of embodiment 17, wherein the first color and the different second color allow a device to determine a location of the plunger assembly within the barrel.

Embodiment 20. The safety syringe of embodiment 19, wherein the first color and the different second color allow the device to determine a volume of a medicinal fluid within the barrel of the safety syringe.

It will become apparent to those of ordinary skill in the art upon reading this specification in relation to the accompanying drawings that various other modifications and alterations can also be made to the embodiments disclosed here, and it is intended that the scope of the invention be limited only by the broadest interpretation of the appended claims to which the inventors are legally entitled.

What is claim is:

1. A safety syringe for medical use, comprising:
an injection-molded body comprising a tubular barrel having a cylindrical inside wall, a proximal end, and a distal end, a cylindrical nose comprising a needle holder comprising a needle coupled to and projecting forwardly from the needle holder at the distal end of the barrel, a finger flange projecting transversely from the barrel near the proximal end, a cylindrical collar projecting rearwardly from the finger flange to the proximal end of the barrel, and two upright wings disposed and extending forwardly from the finger flange in a parallel and spaced-apart relationship above the barrel;
a plunger assembly comprising a plunger handle having a proximal end and a distal end, the proximal end having a plunger handle end cap and the distal end having an elastomeric plunger seal insertable into the barrel through a rearwardly facing opening in the cylindrical collar to slidably engage the cylindrical inside wall of the barrel;
a cylindrical elastomeric bushing frictionally engaging an outside wall of the cylindrical collar;
a selectively removable plunger cap frictionally engaging the cylindrical elastomeric bushing; and
a needle safety shield disposed between the two upright wings and slidably aligned with the barrel.

2. The safety syringe of claim 1, wherein the needle safety shield comprises an activation handle having a proximal end and a distal end, the activation handle slidably engaging the barrel and having an upwardly facing touch surface that facilitates application of manual pressure by a user to initiate forward sliding movement of the needle safety shield relative to the barrel.

3. The safety syringe of claim 2, wherein the forward sliding movement of the needle safety shield repositions a cylindrical needle guard disposed at the distal end of the needle safety shield from a first position surrounding the cylindrical nose of the barrel during storage and use of the safety syringe to a second position guarding a needle tip of the needle following use of the safety syringe.

4. The safety syringe of claim 3, wherein each of the two upright wings comprises an inwardly projecting, longitudinally extending guide rail to which the activation handle of the needle safety shield slidably engages.

5. The safety syringe of claim 4, wherein each longitudinally extending guide rail is cooperatively sized and aligned with a channels disposed on opposing sides of the activation handle of the needle safety shield, and wherein each of the channels slidably engages the respective guide rail.

6. The safety syringe of claim 5, wherein each support and guide rail is integrally molded as part of one of the two upright wings from which it projects.

7. The safety syringe of claim 1, wherein the finger flange comprises a window.

8. The safety syringe of claim 7, wherein the window is cooperatively sized and aligned with an activation handle of the needle safety shield that is disposed between the two upright wings.

9. The safety syringe of claim 7, wherein the window comprises a first side that is contiguous with a proximal end portion of an inwardly facing wall of each of the two upright wings.

10. The safety syringe of claim 9, wherein the window has a top section that bridges and provides structural support for the two upright wings.

11. The safety syringe of claim 1, wherein at least one of the two upright wings comprises a substantially flat, outwardly facing indicia display surface configured to bear indicia related to volumetric measurements of a fluid disposed inside the syringe.

12. The safety syringe of claim 11, wherein each of the two upright wings comprises a substantially flat, outwardly facing surface bearing indicia related to volumetric measurements of a fluid disposed inside the syringe.

13. The safety syringe of claim 1, wherein the two upright wings comprise one or more interference elements molded on inwardly facing surfaces of the two upright wings to restrict sliding movement of the needle safety shield relative to the barrel.

14. The safety syringe of claim 13, wherein each of the one or more interference elements comprise a ramp having a forwardly facing stop shoulder.

15. The safety syringe of claim 14, wherein the needle safety shield comprises a rearwardly facing stop surface that engages forwardly facing stop shoulders of the ramps to prevent subsequent rearward movement of the needle safety shield relative to the barrel following repositioning of the needle safety shield from a first position to a second position.

16. The safety syringe of claim 1, wherein a cylindrical needle guard disposed at the distal end of the needle safety shield surrounds the cylindrical nose of the barrel during storage and use of the safety syringe, and wherein the cylindrical needle guard envelops a needle tip of the needle following use of the safety syringe.

17. The safety syringe of claim 1, wherein the cylindrical collar comprises a first color, and wherein the plunger assembly comprises a second color that is different from the first color.

18. The safety syringe of claim 17, wherein the cylindrical collar is white, and wherein the plunger assembly is blue.

19. The safety syringe of claim 17, wherein the first color and the different second color allow a device to determine a location of the plunger assembly within the barrel.

20. The safety syringe of claim 19, wherein the first color and the different second color allow the device to determine a volume of a medicinal fluid within the barrel of the safety syringe.
